(19) United States
(12) Patent Application Publication
(10) Pub. No.: US 2014/0234366 A1
(43) Pub. Date: Aug. 21, 2014

(54) COMPOSITION FOR PREVENTING OR TREATING ALLERGIC DISEASE OR MAJOR DEPRESSIVE DISORDER (DEPRESSION)

(71) Applicant: Yoshiteru Shimoide, Kagoshima-shi (JP)

(72) Inventor: Yoshiteru Shimoide, Kagoshima-shi (JP)

(21) Appl. No.: 13/822,864

(22) PCT Filed: Feb. 21, 2013
(86) PCT No.: PCT/JP2013/000969

§ 371 Date: Mar. 13, 2013

(30) Foreign Application Priority Data
Feb. 23, 2012 (JP) PCT/JP2012/001231

(51) Int. Cl. A61K 36/756; A61K 36/539; A61K 36/744; A61K 36/258; A61K 36/481; A61K 36/284; A61K 36/233; A61K 36/232; A61K 36/71; A61K 36/752; A61K 36/9068; A61K 36/725; A61K 36/484; A61K 36/076; A61K 36/54; A61K 36/718

(52) U.S. Cl. CPC A61K 36/756; A61K 36/718; A61K 36/539; A61K 36/744; A61K 36/258; A61K 36/481; A61K 36/284; A61K 36/233; A61K 36/232; A61K 36/71; A61K 36/752; A61K 36/9068; A61K 36/725; A61K 36/484; A61K 36/076; A61K 36/54
USPC 424/195.15; 424/745; 424/728

(57) ABSTRACT

The invention aims to provide a composition for treating atopic dermatitis, bronchial asthma, allergic rhinitis or major depressive disorder (depression), comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components. Provided is a composition for preventing or treating an allergic disease or major depressive disorder, comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components.

### TECHNICAL FIELD

The present invention relates to a composition including a kampo combination drug, for preventing or treating atopic dermatitis, bronchial asthma, allergic rhinitis or major depressive disorder (depression).

### BACKGROUND ART

A kampo medicine refers to a prescription containing a combination of crude drugs, for treating a disease by conditioning the whole body of the patient. In a kampo medicine, crude drugs are chosen and prescribed based on patient's symptoms (sho in oriental medicine).

For atopic dermatitis, bronchial asthma and allergic rhinitis, therapies with various drugs have been tried, but few therapies can lead to cure or complete remission at high rates. Some kampo medicines have been said to be effective for these diseases, but there were no clear evidences that these medicines have a therapeutic effect for the above disease. Therefore, it is considered in the art that no kampo medicine cures the above diseases. For major depressive disorder (depression), various medicines have been said to be effective, but with them, remission rates are about 30%, and even in the cases of remission, recurrence is often observed.

Formula antidoti coptidis (Orengedokuto) is composed of four crude drugs of Copridis Rhizoma (oren), Scutellaria Root (ogon), Phellodendron Bark (obaku) and Gardenia Fruit (sanshishi), and said to be effective for gastrointestinal diseases, such as peptic ulcer and gastritis, hot flash, giddiness, neurosis, insomnia, dermatopathia, cutanous pruritus, and the like (see Patent Literatures 1 and 2). Formula repletionis animalis et supletionis medii (Hochuekkito) is comprised of ten crude drugs of Ginseng Root (ninjin), Astragalus Root (ogi), Atractylodes Lancea Rhizome (sojutsu) or Atractylodis Rhizoma (byakujutsu), Bupleurum Root (saiko), Japanese Angelica Root (toki), Cimicifuga Rhizome (shoma), Citrus Unshiu Peel (chinpi), Ginger Rhizome (shokyo), Jujube Fruit (taiso) and Glycyrrhiza Root (kanzo), and said to improve gastrointestinal function to help restore physical strength, and to be effective for physical fatigue, anorexia, weak digestion, summer emaciation, complicated and prolonged cold, hemorrhoids, or when being weak in physical strength, such as during or after sickness, or after operation (Patent Literature 3). Formula glycyrrhizae atractylodis cinnamomi hoelen (Ryokeijutsukanto) is composed of four crude drugs of Hoelen (bukuryo), Atractylodes Lancea Rhizome (sojutsu) or Atractylodis Rhizoma (byakujutsu), Cinnamon Bark (keihi) and Glycyrrhiza Root (kanzo), and said to be effective for improving conditions with giddiness and orthostatic dizziness. Kampo medicines such as Formula repletionis animalis et supletionis medii (hochuekkito) have been also reported to have immunostimulation ability (Patent Literature 4).

Effectiveness of kampo medicines for atopic dermatitis, bronchial asthma, allergic rhinitis and major depressive disorder have been reported (Non Patent Literatures 1-3), but it has not been reported that a mixture of kampo medicines leads to cure or complete remission.

### CITATION LIST

### Patent Literature

PTL 1: JP Patent Publication (Kokai) No. 2003-113108
PTL 2: JP Patent Publication (Kokai) No. 2004-091408
PTL 3: WO2007/077606
PTL 4: JP Patent Publication (Kokai) No. 2003-306442

### Non Patent Literature

NPL 1: Egashira Y, nagano H, et al: Ann NY Acad Sci. 1993, 685, 580-583.
NPL 2: Ikeda Y et al: Jpn J Pharmacol, 90(4): 328-336, 2002
NPL 3: Mizoguchi K et al: Pharmacol Biochem Behav, 75(2): 419-425, 2003

### SUMMARY OF INVENTION

### Technical Problem

The present invention aims to provide a composition for treating atopic dermatitis, bronchial asthma, allergic rhinitis or major depressive disorder, comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components.

### Solution to Problem

Certain kampo medicines have been reported to be effective for allergic diseases. Nevertheless, the kampo medicines were prescribed for symptoms (sho) of each patient, but not for a particular disease. Sho does not represent a particular disease, but refers to general state of the patient including physical strength, constitution, and symptoms of the patient. Moreover, many of individual crude drugs contained in kampo medicines were not clear on their pharmacologic effects. Therefore, a kampo medicine said to be effective for an allergic disease was that for the sho of the patient having a symptom, for example, itchy skin etc., similar to the allergic disease, but not that effective for treating the allergic disease as a disease. In fact, there was little evidence demonstrating with a pharmacologic effect that a kampo medicine can be used as a therapeutic drug for a certain disease.

The present inventors studied diligently for kampo medicines effective for patients with a specific disease, atopic dermatitis, bronchial asthma, allergic rhinitis or major depressive disorder, without being based on the sho of the patient. As a result, they found that a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) cures these diseases in high efficiency by suppressing the activation of macrophage lineage cells in the living body. By administering these mixture to a patient and examining the effects, the present inventors found that the above mixture can be used as a preventive or therapeutic drug for a specific disease, atopic dermatitis, bronchial asthma, allergic rhinitis or major depressive disorder, and completed the present invention on a therapeutic composition comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components.

Thus, the present invention is as follows:

[1] a composition for preventing or treating an immune disease, comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active ingredient;

[2] the composition for preventing or treating an allergic disease, being the composition for preventing or treating an immune disease according to [1], wherein the immune disease is an allergic disease;

[3] the composition for preventing or treating an allergic disease according to [2], wherein the allergic disease is atopic dermatitis;

[4] the composition for preventing or treating an atopic dermatitis, being the composition for preventing or treating an allergic disease according to [3], comprising a mixture of Formula antidoti coptidis (orengedokuto) and Formula repletionis animalis et supletionis medii (hochuekkito) as active components, used for treating atopic dermatitis showing inflammation in trunk or limbs, but not in face, ear or head;

[5] the composition for preventing or treating an atopic dermatitis, being the composition for preventing or treating an allergic disease according to [3], comprising a mixture of Formula antidoti coptidis (orengedokuto) and Formula glycyrrhizae atractylodis cinnamomi hoelen (ryokeijutsukanto) as active components, used for treating atopic dermatitis showing inflammation in face, ear or head, with or without inflammation in neck and trunk thereabove or limbs;

[6] the composition for preventing or treating an allergic disease, comprising a mixture of Formula antidoti coptidis (orengedokuto) and Formula glycyrrhizae atractylodis cinnamomi hoelen (ryokeijutsukanto) as active components according to [2], wherein the allergic disease is selected from the group consisting of bronchial asthma and allergic rhinitis;

[7] the composition for treating major depressive disorder (depression), being the composition for treating immune disease, comprising a mixture of Formula antidoti coptidis (orengedokuto) and Formula glycyrrhizae atractylodis cinnamomi hoelen (ryokeijutsukanto) as active components according to [1], wherein the immune disease is major depressive disorder (depression).

### Advantageous Effects of Invention

The composition comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components according to the present invention can be used for preventing or treating a specific disease, i.e. allergic disease, which is atopic dermatitis, bronchial asthma or allergic rhinitis, or major depressive disorder (depression), without being based on sho.

### DESCRIPTION OF EMBODIMENTS

The present invention will be explained in detail below.

A composition of the present invention is a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto), comprising Formula antidoti coptidis (orengedokuto) and Formula repletionis animalis et supletionis medii (hochuekkito) or Formula antidoti coptidis (orengedokuto) and Formula glycyrrhizae atractylodis cinnamomi hoelen (ryokeijutsukanto) as active components.

Formula antidoti coptidis (orengedokuto) is a kampo medicine prepared by mixing crude drugs of Copridis Rhizoma (oren), Scutellaria Root (ogon), Phellodendron Bark (obaku) and Gardenia Fruit (sanshishi). In a daily amount for adult, mixed weights of the dried crude drugs are for example, Copridis Rhizoma 1.5 g-2.0 g, Scutellaria Root 3.0 g, Phellodendron Bark 1.5 g-3.0 g, Gardenia Fruit 2.0 g-3.0 g.

Hereinbelow, Copridis Rhizoma, Scutellaria Root, Phellodendron Bark and Gardenia Fruit will be explained, but they are not limited to those described below. Copridis Rhizoma refers to dried rhizome (without most of the root) of Coptis japonica Makino or other species in the same genus in the family of Ranunculaceae. Scutellaria Root refers to dried root (without periderm) of Scutellaria baicalensis Georgi in the family of Labiatae. Phellodendron Bark refers to dried bark (without periderm (cork-cortex)) of Phellodendron amurense RUPR, or other species in the same genus in the family of Rutaceae. Other species in the same genus include, for example, Phellodendron chinense Schneider. Gardenia Fruit refers to dried fruits of Gardenia jasminoides ellis or other species in the same genus in the family of Rubiaceae.

Formula antidoti coptidis (orengedokuto) can be prepared by obtaining the extract preparation of the four crude drugs and mixing them according to the mixed weights described above. Or, it can be prepared by putting the four dried crude drugs in 600 ml of hot water according to the mixed weights described above, and boiling it down (concentrating) to 300 ml for one hour, yielding a daily amount in the 300 ml. Two kampo medicines, 100 ml each a time, are mixed to 200 ml a time to be administered three times a day before every meal. The two kampo medicines, 150 ml each a time, can be mixed to 300 ml a time to be administered twice a day before morning and evening meals.

Commercial medicines are also available. Commercial medicines include Ohsugi Formula antidoti coptidis (orengedokuto) extract T tablet (Ohsugi Pharmaceutical Co., Ltd), Kracie Formula antidoti coptidis (orengedokuto) extract tablet (Kracie Pharma, Ltd.), Kotaro Formula antidoti coptidis (orengedokuto) extract fine granule (Kotaro pharmaceutical Co., Ltd.), Sakamoto Formula antidoti coptidis (orengedokuto) extract granule-S (Sakamoto Kanpoh Pharmaceutical Co., Ltd.), Tsumura Formula antidoti coptidis (orengedokuto) extract granule (ethical) (TSUMURA & CO.), JPS Formula antidoti coptidis (orengedokuto) extract granule (dispensing) (JPS Pharmaceutical Co., Ltd.).

Formula repletionis animalis et supletionis medii (hochuekkito) is a kampo medicine prepared by mixing crude drugs of Ginseng Root (ninjin), Astragalus Root (ogi), Atractylodes Lancea Rhizome (sojutsu) or Atractylodis Rhizoma (byakujutsu), Bupleurum Root (saiko), Japanese Angelica Root (toki), Cimicifuga Rhizome (shoma), Citrus Unshiu Peel (chinpi), Ginger Rhizome (shokyo), Jujube Fruit (taiso) and Glycyrrhiza Root (kanzo). In a daily amount for adult, mixed weights of the dried crude drugs are for example, Ginseng Root 4.0 g, Astragalus Root 3.0 g, Atractylodes Lancea Rhizome or Atractylodis Rhizoma 4.0 g, Bupleurum Root 2.0 g, Japanese Angelica Root 3.0 g, Cimicifuga Rhizome 1.0 g, Citrus Unshiu Peel 2.0 g, Ginger Rhizome 2.0 g, Jujube Fruit 2.0 g, Glycyrrhiza Root 1.5 g.

Hereinbelow, Ginseng Root, Astragalus Root, Atractylodes Lancea Rhizome or Atractylodis Rhizoma, Bupleurum Root, Japanese Angelica Root, Cimicifuga Rhizome, Citrus Unshiu Peel, Ginger Rhizome, Jujube Fruit and Glycyrrhiza Root will be explained, but they are not limited to those described below. Ginseng Root refers to root (without rootlet) or lightly steamed and dried root of Panax ginseng C. A. Meyer in the family of Araliaceae. Astragalus Root is root of Astragalus membranaceus Bunge or other species in the same genus in the family of Leguminosae. Other species in the same genus include A. mongholicus Bunge. Atractylodes Lancea Rhizome refers to dried rhizome of Atractylodes lancea De Candolle or its varieties in the family of Compositae. The varieties include A. lancea DC. var. chinensis Kitamura. Atractylodis Rhizoma refers to rhizome (usually without periderm) of Atractylodes japonica Koidzumi (wabyakujutsu) or rhizome of Atractylodes ovata De Candolle (karabyakujutsu). Bupleurum Root refers to root of Bupleurum falcatum L. or its varieties in the family of Umbelliferae. The varieties of Bupleuri Radix include Bupleurum chinense DC (kitasaiko) and B. scorzoneraefolium Willd (minamisaiko), and Stellaria dichotoma L. var. lanceolata Bunge in the family of Caryophylaceae. Japanese Angelica Root refers to root (usually steamed root) of Angelica acutiloba Kitagawa or other closely related species in the family of Umbelliferae. Other closely related species include A. acutiloba Kitagawa subsp, iwatwnsis Kitagawa and A. sinensis (Oliv.) Diels. Cimicifuga Rhizome refers to rhizome of Cimicifuga simplex Woiinskjord or other species in the same genus in the family of Ranunculaceae. Other species in the same genus include C. dahurica (Turcz.) Maxim. (kitashoma) and C. heracleifolia Komarov (kanshoma). Citrus Unshiu Peel refers to dried mature pericarp of Citrus unshiu Markov. or other closely related species in the family of Rutaceae. Other closely related species include ponkan and orange. Ginger Rhizome refers to rhizome of Zingiber officinale Roscoe in the family of Zingiberaceae. Jujube Fruit refers to dried mature fruit of Zizyphus jujuba Miller var. inermis (Bunge) Rehder or other closely related species in the family of Rhamnaceae. Glycyrrhiza Root refers to root and sarmentum of Glycyrrhiza uralensis Fischer, Glycyrrhiza glabra L. and other species in the same genus in the family of Leguminosae.

Formula repletionis animalis et supletionis medii (hochuekkito) can be prepared by obtaining the extract preparation of the ten crude drugs and mixing them according to the mixed weights described above. Or, it can be prepared by putting the ten dried crude drugs in 600 ml of hot water according to the mixed weights described above, and boiling it down for one hour to concentrate into 300 ml, yielding a daily amount in the 300 ml. Commercial medicines are also available. Commercial medicines include Kracie Formula repletionis animalis et supletionis medii (hochuekkito) extract fine granules (Kracie Pharma, Ltd.), Sanwa Formula repletionis animalis et supletionis medii (hochuekkito) A extract tablet (Sanwa Shoyaku), Tsumura Formula repletionis animalis et supletionis medii (hochuekkito) extract granules (TSUMURA & CO.), Formula repletionis animalis et supletionis medii (hochuekkito) extract tablet “Kotaro” (Kotaro pharmaceutical Co., Ltd.), Rohto Formula repletionis animalis et supletionis medii (hochuekkito) tablet (ROHTO Pharmaceutical Co., Ltd.), and JPS Formula repletionis animalis et supletionis medii (hochuekkito) extract granules (dispensing) (JPS Pharmaceutical Co., Ltd.).

Formula glycyrrhizae atractylodis cinnamomi hoelen (ryokeijutsukanto) is a kampo medicine prepared by mixing crude drugs of Hoelen (bukuryo), Atractylodes Lancea Rhizome (sojutsu) or Atractylodis Rhizoma (byakujutsu), Cinnamon Bark (keihi) and Glycyrrhiza Root (kanzo). In a daily amount for adult, mixed weights of the dried crude drugs are for example, Hoelen 4 g-6 g, Atractylodes Lancea Rhizome or Atractylodis Rhizoma 2 g-3 g, Cinnamon Bark 3 g-4 g, Glycyrrhiza Root 1 g-2 g.

Hereinbelow, Hoelen and Cinnamon Bark will be explained, but they are not limited to those described below. Atractylodes Lancea Rhizome or Atractylodis Rhizoma and Glycyrrhiza Root are as above.

Hoelen refers to dried sclerotium (as it is or without most of the outer layer) of Poria cocos Wolf in the family of Polyporaceae. Cinnamon Bark refers to dried bark of Cinnamomum cassia Blume or other species in the same genus in the family of Lauraceae.

Formula glycyrrhizae atractylodis cinnamomi hoelen (ryokeijutsukanto) can be prepared by obtaining the extract preparation of the four crude drugs and mixing them according to the mixed weights described above. Or, it can be prepared by putting the four dried crude drugs in 600 ml of hot water according to the mixed weights described above, and boiling it down to 300 ml for one hour, yielding a daily amount in the 300 ml. Commercial medicines are also available. Commercial medicines include Kracie Formula glycyrrhizae atractylodis cinnamomi hoelen (ryokeijutsukanto) extract fine granule (Kracie Pharma, Ltd.), Kotaro Formula glycyrrhizae atractylodis cinnamomi hoelen (ryokeijutsukanto) extract fine granule (Kotaro pharmaceutical Co., Ltd.), Tsumura Formula glycyrrhizae atractylodis cinnamomi hoelen (ryokeijutsukanto) extract granule (TSUMURA & CO.), Ohsugi Formula glycyrrhizae atractylodis cinnamomi hoelen (ryokeijutsukanto) extract TG (Ohsugi Pharmaceutical Co., Ltd), JPS Formula glycyrrhizae atractylodis cinnamomi hoelen (ryokeijutsukanto) extract granule (dispensing) (JPS Pharmaceutical Co., Ltd.).

The extracts of crude drugs included in Formula antidoti coptidis (orengedokuto), Formula repletionis animalis et supletionis medii (hochuekkito) or the Formula glycyrrhizae atractylodis cinnamomi hoelen (ryokeijutsukanto) can be prepared as powder, for example, by extracting the crude drugs with water or heated water, ethanol, acetic acid, or the like, and drying by spray-dry or lyophilization. The powders are mixed to prepare Formula antidoti coptidis (orengedokuto), Formula repletionis animalis et supletionis medii (hochuekkito) or Formula glycyrrhizae atractylodis cinnamomi hoelen (ryokeijutsukanto). These are mixed to prepare a mixture.

A mixture of Formula antidoti coptidis (orengedokuto) and Formula repletionis animalis et supletionis medii (hochuekkito) may be obtained by mixing 1.3 g-2.71 g of powdered Formula antidoti coptidis (orengedokuto) dry extract and 3.57 g-7 g of powdered Formula repletionis animalis et supletionis medii (hochuekkito) dry extract for an adult daily dose. In this case, the two kampo medicines, 100 ml each a time, are mixed to 200 ml a time to be administered three times a day before every meal. The two kampo medicines, 150 ml each a time, can be mixed to 300 ml a time to be administered twice a day before morning and evening meals. When commercial kampo medicine extracts are used, a commercial Formula antidoti coptidis (orengedokuto) extract and a commercial Formula repletionis animalis et supletionis medii (hochuekkito) extract may be divided into aliquots of one-third of the indicated daily amount and mixed to be administered three times a day before meal. Also, the commercial Formula antidoti coptidis (orengedokuto) extract and the commercial Formula repletionis animalis et supletionis medii (hochuekkito) extract may be divided into aliquots of half the indicated daily amount and mixed to be administered twice a day before morning and evening meals. A mixture of Formula antidoti coptidis (orengedokuto) and Formula glycyrrhizae atractylodis cinnamomi hoelen (ryokeijutsukanto) may be obtained by mixing 1.3 g-2.71 g of powdered Formula antidoti coptidis (orengedokuto) dry extract and 1.5 g-7 g of powdered Formula glycyrrhizae atractylodis cinnamomi hoelen (ryokeijutsukanto) dry extract for an adult daily dose. In this case, the two kampo medicines, 100 ml each a time, are mixed to 200 ml a time to be administered three times a day before every meal. The two kampo medicines, 150 ml each a time, can be mixed to 300 ml a time to be administered twice a day before morning and evening meals. When commercial kampo medicine extracts are used, a commercial Formula antidoti coptidis (orengedokuto) extract and a commercial Formula glycyrrhizae atractylodis cinnamomi hoelen (ryokeijutsukanto) extract may be divided into aliquots of one-third of the indicated daily amount and mixed to be administered three times a day before meal. Also, the commercial Formula antidoti coptidis (orengedokuto) extract and the commercial Formula glycyrrhizae atractylodis cinnamomi hoelen (ryokeijutsukanto) extract may be divided into aliquots of half the indicated daily amount and mixed to be administered twice a day before morning and evening meals. A composition according to the present invention can be obtained by adding a suitable excipient, adjuvant or the like for common pharmaceutical use to the mixed extracts and formulating them as an oral preparation such as powder, granule, tablet, capsule, solution, syrup according to a usual method for pharmaceutical production. In addition, a binding agent, disintegrating agent, surfactant, flavoring agent, or perfume may be added as appropriate. Excipients include, for example, starch, dextrin, milk sugar, refined sugar, mannite, crystal cellulose, silicic anhydride, and the like.

A composition according to the present invention contains Formula antidoti coptidis (orengedokuto) and Formula repletionis animalis et supletionis medii (hochuekkito) or Formula antidoti coptidis (orengedokuto) and Formula glycyrrhizae atractylodis cinnamomi hoelen (ryokeijutsukanto) usually at around 0.1-100% (w/w) of the total composition, depending on its dosage form. The dose of a composition according to the invention for a patient may be administered once or several times a day for several days to several tens of months, under consideration of the age of the patient.

A mixture of Formula antidoti coptidis (orengedokuto) and Formula repletionis animalis et supletionis medii (hochuekkito) and a mixture of Formula antidoti coptidis (orengedokuto) and Formula glycyrrhizae atractylodis cinnamomi hoelen (ryokeijutsukanto) according to the invention can reduce activities of macrophage linage immunocytes in the living body, and suppress inflammatory reactions and allergic reactions. A composition containing the mixture can be used for treating an allergic disease such as atopic dermatitis, bronchial asthma and allergic rhinitis, or major depressive disorder. Major depressive disorder is also termed depression.

The mixture of Formula antidoti coptidis (orengedokuto) and Formula repletionis animalis et supletionis medii (hochuekkito) and the mixture of Formula antidoti coptidis (orengedokuto) and Formula glycyrrhizae atractylodis cinnamomi hoelen (ryokeijutsukanto) can be used as an agent for preventing or treating atopic dermatitis. For atopic dermatitis with inflammation of head and face, ear or head, with or without inflammation of trunk or limbs, a mixture of Formula antidoti coptidis (orengedokuto) and Formula glycyrrhizae atractylodis cinnamomi hoelen (ryokeijutsukanto) may be administered. For atopic dermatitis with inflammation for inflammation of trunk or limbs, but without inflammation of face, ear or head, a mixture of Formula antidoti coptidis (orengedokuto) and Formula repletionis animalis et supletionis medii (hochuekkito) may be administered.

As to the dose of commercial ethical kampo extract preparations with a daily dose of 7.5 g, for example, for an adult, a mixture of 2.5 g each of kampo medicines is orally administered three times a day before meal. Depending on the reaction of each patient, the mixture is administered until the symptoms and skin inflammation of the patient are resolved. For a patient younger than 17 years old, the mixture may be administered according to the age-specific therapeutic dose shown in Table 1.

TABLE 1

| | | Ratio to | | Dose for 15 | Dose for 30 |
|---|---|---|---|---|---|
| Age after birth | | adult dose | Daily dose g | days g | days g |
| 1-7 | days | 1.30% | 0.0975 | 1.4625 | 2.925 |
| 7-15 | days | 2.00% | 0.15 | 2.25 | 4.5 |
| 15-20 | days | 2.70% | 0.2025 | 3.0375 | 6.075 |
| 20-30 | days | 4.00% | 0.3 | 4.5 | 9 |
| 30-40 | days | 7.00% | 0.525 | 7.875 | 15.75 |
| 40-60 | days | 8.00% | 0.6 | 9 | 18 |
| 60-100 | days | 10.00% | 0.75 | 11.25 | 22.5 |
| 100-200 | days | 12.00% | 0.9 | 13.5 | 27 |
| 200 days-1 year | | 20.00% | 1.5 | 22.5 | 45 |
| 1-1.5 | years | 25% | 1.875 | 28.125 | 56.25 |
| 1.5-2 | years | 26% | 1.95 | 29.25 | 58.5 |
| 2-2.5 | years | 28% | 2.1 | 31.5 | 63 |
| 2.5-3 | years | 30% | 2.25 | 33.75 | 67.5 |
| 3-3.5 | years | 32% | 2.4 | 36 | 72 |
| 3.5-4 | years | 34% | 2.55 | 38.25 | 76.5 |
| 4-4.5 | years | 36% | 2.7 | 40.5 | 81 |
| 4.5-5 | years | 38% | 2.85 | 42.75 | 85.5 |
| 5-5.5 | years | 40% | 3 | 45 | 90 |
| 5.5-6 | years | 42% | 3.15 | 47.25 | 94.5 |
| 6-6.5 | years | 44% | 3.3 | 49.5 | 99 |
| 6.5-7 | years | 46% | 3.45 | 51.75 | 103.5 |
| 7-7.5 | years | 48% | 3.6 | 54 | 108 |
| 7.5-8 | years | 50% | 3.75 | 56.25 | 112.5 |
| 8-8.5 | years | 52% | 3.9 | 58.5 | 117 |
| 8.5-9 | years | 54% | 4.05 | 60.75 | 121.5 |
| 9-9.5 | years | 56% | 4.2 | 63 | 126 |
| 9.5-10 | years | 58% | 4.35 | 65.25 | 130.5 |
| 10-10.5 | years | 60% | 4.5 | 67.5 | 135 |
| 10.5-11 | years | 62% | 4.65 | 69.75 | 139.5 |
| 11-11.5 | years | 64% | 4.8 | 72 | 144 |
| 11.5-12 | years | 66% | 4.95 | 74.25 | 148.5 |
| 12-12.5 | years | 68% | 5.1 | 76.5 | 153 |
| 12.5-13 | years | 70% | 5.25 | 78.75 | 157.5 |
| 13-13.5 | years | 72% | 5.4 | 81 | 162 |
| 13.5-14 | years | 74% | 5.55 | 83.25 | 166.5 |
| 14-14.5 | years | 78% | 5.85 | 87.75 | 175.5 |
| 14.5-15 | years | 80% | 6 | 90 | 180 |
| 15-17 | years | 90% | 6.75 | 101.25 | 202.5 |

| | | | | |
|---|---|---|---|---|
| 17 years or older | 100% | 7.5 | 112.5 | 225 |

After the treatment with a composition according to the invention, recurrences of atopic dermatitis are usually not observed. In rare cases of recurrence, inflammation is resolved by the medication for shorter-term than the first treatment, for example, 2-3 weeks. Moreover, the cure rate of atopic dermatitis with a mixture according to the invention is not less than 88%.

A mixture of Formula antidoti coptidis (orengedokuto) and Formula glycyrrhizae atractylodis cinnamomi hoelen (ryokeijutsukanto) can be used for preventing or treating an immune disease such as allergic diseases. Allergic diseases include bronchial asthma and allergic rhinitis. Patients with atopic dermatitis are complicated with bronchial asthma and allergic rhinitis at high rates. A composition according to the invention can be used for preventing or treating bronchial asthma and allergic rhinitis in the atopic dermatitis patient with complications. Moreover, the composition can be used for preventing or treating bronchial asthma and allergic rhinitis of the patients other than the atopic dermatitis patient.

As to commercial ethical kampo extract preparation with a daily dose of 7.5 g, for example, for an adult, a mixture of Formula antidoti coptidis (orengedokuto) 2.5 g and Formula glycyrrhizae atractylodis cinnamomi hoelen (ryokeijutsukanto) is orally administered three times a day before meal. The time to the resolution of symptoms was 16.2 days in 200 cases of patients with moderate or severe bronchial asthma. In 123 cases of moderate or more severe allergic rhinitis, the time to the resolution of symptoms was 9.3 days. In bronchial asthma and allergic rhinitis patients, symptoms developed at the time of upper respiratory infection. The symptoms were resolved without recurrence, as the upper respiratory infection healed. The dosage period was 1-14 weeks for either disease. For atopic dermatitis, the administration is preferably continued for about eight weeks or more from the time the skin symptoms, i.e. inflammation and itching are resolved. For bronchial asthma, the administration is preferably continued for about eight weeks or more from the time symptoms such as cough and shortness of breath are completely resolved. For allergic rhinitis, the administration is preferably continued for about four weeks or more from the time the symptoms are completely resolved.

The cure rate of allergic disease with a mixture according to the invention is not less than 93%.

A mixture of Formula antidoti coptidis (orengedokuto) and Formula glycyrrhizae atractylodis cinnamomi hoelen (ryokeijutsukanto) can be used for preventing or treating major depressive disorder. In other words, a mixture of Formula antidoti coptidis (orengedokuto) and Formula glycyrrhizae atractylodis cinnamomi hoelen (ryokeijutsukanto) can be used as a composition having antidepressant effect, or as antidepressant. Various ways of classifying major depressive disorder have been suggested. In the present invention, major depressive disorder refers to diseases, for example, classified as “major depressive disorder” related disorders in “International Classification of Diseases, Injuries and Causes of Death. 10th version” (ICD-10) by World Health Organization, or “major depressive disorder” as evaluated by multiaxial diagnosis according to DSM-IV in “Diagnostic Statistic Manual of Mental Disorders” (DSM) by American Psychiatric Association (APA). In the present invention, major depressive disorder includes major depressive disorder-like symptoms and depressive symptoms.

The inventors of the present application studied individual immunological differences after the interferon therapy for chronic hepatitis C, between the cases where the therapy was effective and the cases where the therapy was not effective. In twenty and several more cases, the interferon therapy was so successful that hepatitis C virus disappeared and the liver functions became normal. In three cases among them, immunological activities, such as soluble CD35 (sCD35) antigen activities and macrophage activities, remained high after the disappearance of the virus. The followups of the three cases revealed that the patients were suffering from major depressive disorder, a side effect of interferon, due to the interferon administration. Thus, the inventors found that major depressive disorder was caused by activation of immunity, i.e., imbalance of cytokines due to immunological inflammation with macrophages playing a central role. The inventors also found that patients with allergic diseases were complicated with major depressive disorder at a high rate; 88.8% of patients with major depressive disorder were also suffering from an allergic disease. Moreover, the inventors found that treating an allergic disease with a kampo medicine(s) ameliorates major depressive disorder. Thus, it can be said that major depressive disorder is a disease caused by immunological inflammation. In addition, the inventors found that among the mixtures of kampo medicines used for amelioration of major depressive disorder, only mixtures of Formula antidoti coptidis (orengedokuto) and Formula glycyrrhizae atractylodis cinnamomi hoelen (ryokeijutsukanto) ameliorated major depressive disorder, and that major depressive disorder was completely ameliorated by a mixture of the above kampo medicines even in a major depressive disorder patient without history of an allergic disease. These results indicate that major depressive disorder is an immune disease. Macrophage linage cells such as dendritic cells are involved in inflammation of atopic dermatitis, bronchial asthma and allergic rhinitis. Activity of CD35 is involved in development of major depressive disorder. Based on these, a mixture of Formula antidoti coptidis (orengedokuto) and Formula glycyrrhizae atractylodis cinnamomi hoelen (ryokeijutsukanto) according to the invention are capable of suppressing activities of macrophage linage cells, and capable of preventing or treating major depressive disorder by suppressing activities of soluble CD35. As to the doses of commercial ethical kampo extracts of Formula antidoti coptidis (orengedokuto) with a daily dose of 7.5 g and Formula glycyrrhizae atractylodis cinnamomi hoelen (ryokeijutsukanto) with a daily dose of 7.5 g for an adult, a mixture of Formula antidoti coptidis (orengedokuto) 2.5 g and Formula glycyrrhizae atractylodis cinnamomi hoelen (ryokeijutsukanto) 2.5 g is orally administered three times a day before meal. For major depressive disorder, the administration is preferably continued for about eight weeks or more from the time DSM-IV symptoms are resolved and SDS became under 40.

A composition according to the invention is directed to mammals including human. Mammals other than human include primates such as monkey, rodents such as mouse and rat, sheep, swine, cattle, cat and dog.

Furthermore, a composition according to the invention can be mixed into food or beverage, and used as a food and beverage composition or a feed composition. For this, Formula antidoti coptidis (orengedokuto) and Formula repletionis animalis et supletionis medii (hochuekkito) or Formula antidoti coptidis (orengedokuto) and Formula glycyrrhizae atractylodis cinnamomi hoelen (ryokeijutsukanto), for example, commercial ethical kampo extracts with a human daily dose of 7.5 g, may be mixed at 100 mg/kg into food and beverage, or feed for one ingestion. A composition according to the invention can be used as a food and beverage composition or a feed composition, such as powder, granule, liquid, or paste. Food and beverage include health food and beverage, designated health food and beverage, functional nutritional food and beverage, supplement food and beverage, and the like. Herein, designated health food refers to food taken in diet for a specific health benefit and indicating that the benefit is expected thorough eating it.

Although the present invention will be explained specifically by means of the following examples, it is not limited by the examples.

### EXAMPLE 1

Preparation of Mixture of Formula antidoti coptidis (orengedokuto) and Formula repletionis animalis et supletionis medii (hochuekkito)

In this example, commercial kampo extracts were used. A commercial Formula antidoti coptidis (orengedokuto) extract and a commercial Formula repletionis animalis et supletionis medii (hochuekkito) extract were divided into aliquots of one-third of the indicated daily amounts and mixed to be administered three times a day before meal. The commercial Formula antidoti coptidis (orengedokuto) extract and the commercial Formula repletionis animalis et supletionis medii (hochuekkito) extract can be divided into aliquots of half the indicated daily amount and mixed to be administered twice a day before morning and evening meals.

When treating a subject younger than 17 years old, the extracts were administered according to the age-specific therapeutic dose shown in Table 1, indicated in relative to 100% of the daily therapeutic dose for adult and those equal to or older than 17.

### EXAMPLE 2

Preparation of Mixture of Formula antidoti coptidis (orengedokuto) and Formula glycyrrhizae atractylodis cinnamomi hoelen (ryokeijutsukanto)

In this example, commercial kampo extracts were used. A commercial Formula antidoti coptidis (orengedokuto) extract and a commercial Formula glycyrrhizae atractylodis cinnamomi hoelen (ryokeijutsukanto) extract were divided into aliquots of one-third of the indicated daily amount and mixed to be administered three times a day before meal. The commercial Formula antidoti coptidis (orengedokuto) extract and the commercial Formula glycyrrhizae atractylodis cinnamomi hoelen (ryokeijutsukanto) extract can be divided into aliquots of half the indicated daily amount and mixed to be administered twice a day before morning and evening meals.

When treating a subject younger than 17 years old, the extracts were administered according to the age-specific therapeutic dose shown in Table 1, indicated in relative to 100% of the daily therapeutic dose for adult and those equal to or older than 17.

### EXAMPLE 3

[Treatment of Atopic Dermatitis]

The combination drug of Formula antidoti coptidis (orengedokuto) and Formula repletionis animalis et supletionis medii (hochuekkito) prepared by the method of Example 1 and the combination drug of Formula antidoti coptidis (orengedokuto) and Formula glycyrrhizae atractylodis cinnamomi hoelen (ryokeijutsukanto) prepared by the method of Example 2 were used for the treatment of atopic dermatitis.

Treatment was carried out for 241 AD (atopic dermatitis) sufferers (average age 23.4 years, male:female=90:151, average length of illness 10.5 years). Of these AD sufferers, the degree of severity was most severe in 69 cases (28.6%), severe in 78 cases (32.4%), moderate in 91 cases (37.8%) and mild in 3 cases (1.2%). The degree of severity was judged in accordance with the Guidelines for the Treatment of Atopic Dermatitis issued by the Japanese Ministry of Health, Labour and Welfare in 2005. The treatment using the composition was carried out by oral administration.

For atopic dermatitis patients with inflammation of trunk or limbs, but without inflammation of face, ear or head, the combination drug of Formula antidoti coptidis (orengedokuto) and Formula repletionis animalis et supletionis medii (hochuekkito) was administered. Since the daily dose of Tsumura Formula antidoti coptidis (orengedokuto) extract granule (ethical) (TSUMURA & CO.) and Tsumura Formula repletionis animalis et supletionis medii (hochuekkito) extract granule (ethical) (TSUMURA & CO.) is 7.5 g, a mixture of 2.5 g each of them was administered three times a day before meal.

For atopic dermatitis patients with inflammation of face, ear or head irrespective of inflammation of trunk or limbs, the combination drug of Formula antidoti coptidis (orengedokuto) and Formula glycyrrhizae atractylodis cinnamomi hoelen (ryokeijutsukanto) was administered. Since the daily dose of Tsumura Formula antidoti coptidis (orengedokuto) extract granule (ethical) (TSUMURA & CO.) and Tsumura Formula glycyrrhizae atractylodis cinnamomi hoelen (ryokeijutsukanto) extract granule (ethical) (TSUMURA & CO.) is 7.5 g, a mixture of 2.5 g each of them was administered three times a day before meal.

For a subject who is under 17 years old, dose was determined according to the age-specific therapeutic dose shown in Table 1. Before the treatment was started, allergen for each subject was searched. The allergen positive for each subject was avoided during the treatment.

The effects were that 193 cases were cured completely (80.1%), 23 cases showed a marked improvement (9.5%), 3 cases showed an improvement (1.2%), and 22 cases dropped out (9.1%). Excluding the dropout cases, 88.1% of the 219 cases that could be treated were cured, while 10.5% showed marked improvement. The length of administration till the curing was 11.3 weeks on average (1 to 65 weeks), and the length for treatment was 15.0 weeks (1 to 98.7 weeks).

### EXAMPLE 4

[Treatment of Bronchial Asthma]

[Method]

The combination drug of Formula antidoti coptidis (orengedokuto) and Formula glycyrrhizae atractylodis cinnamomi hoelen (ryokeijutsukanto) prepared by the method of Example 2 was used for the treatment of bronchial asthma. Since the daily dose of Tsumura Formula antidoti coptidis (orengedokuto) extract granule (ethical) (TSUMURA & CO.) and Tsumura Formula glycyrrhizae atractylodis cinnamomi hoelen (ryokeijutsukanto) extract granule (ethical) (TSUMURA & CO.) is 7.5 g, a mixture of 2.5 g each of them was administered three times a day before meal.

The treatment using the composition was carried out for 278 persistent bronchial asthma patients (average age was 43.0 years old, male:female=99:179) whose degree of severity was moderate or severe. Fifty two patients were given standard treatment and 226 patients were treated with kampo drugs. The degree of severity and treatment effects were assessed (GINA Report, 2006. 2008) at the start of treatment and when symptoms stabilized. A patient who had symptom during night which was asthma attack at dawn almost every day was diagnosed as severe, and a patient who had symptom during night which was asthma attack at dawn more than once every week was diagnosed as moderate. The patients were also observed using ACT (Asthma Control Test). In standard treatment, all subjects were treated with inhaled corticosteroids+long acting beta 2-adrenergic agent (ICS+LABA). The treatment with leukotorien antagonist was added depending on the symptom of the patient.

### [Results]

There was no statistical difference in bronchial asthma severity at the start of treatment. After treatment, symptoms completely disappeared in 93.5% (200/214) (dropouts=12) of the kampo group and 26.9% (14/52) of the standard group. The complete remission was 93.5% of the kampo group and 25% of the standard group; thus, treatment by kampo drugs had significant effects in both cases (P<0.01). ACT at the start of treatment and when symptoms became stabilized was 14.7 vs 24.8 in the kampo group and 14.0 vs 20.3 in the standard group, again showing that treatment by kampo drugs was significantly more effective (P<0.01). In the kampo group, symptoms disappeared after 16.2 days (n=200).

Current method for bronchial asthma has been insufficient. The present example showed that the treatment by using kampo drugs resulted in complete remission for almost all patients.

### EXAMPLE 5

[Treatment of Allergic Rhinitis]

The combination drug of Formula antidoti coptidis (orengedokuto) and Formula glycyrrhizae atractylodis cinnamomi hoelen (ryokeijutsukanto) prepared by the method of Example 2 was used for the treatment of allergic rhinitis. The combination drug was prepared by mixing 2.5 g of Tsumura Formula antidoti coptidis (orengedokuto) extract granule (ethical) (TSUMURA & CO.) and 2.5 g of Tsumura Formula glycyrrhizae atractylodis cinnamomi hoelen (ryokeijutsukanto) extract granule (ethical) (TSUMURA & CO.), and was administered three times a day before meal.

The diagnosis of allergic rhinitis was carried out according to Practical Guideline for the Management of Allergic Rhinitis in Japan (authored by the committee drafting Practical Guideline for the Management of Allergic Rhinitis in Japan, published by LIFE SCIENCE CO., LTD.). The subjects were patients who suffered from persistent allergic rhinitis and had symptoms of moderate or severe.

Treatment using the composition of the present invention was carried out for 123 persistent allergic rhinitis patients. The treatment was carried out by oral administration of the composition. When the subject who received treatment exhibited no symptoms and no treatment was needed, it was judged that the complete remission was achieved.

The complete remission was achieved for 93.5% of the patients. The average length until the symptoms disappeared was 9.3 days. No adverse effects were observed for patient who received the treatment, and recurrence was not observed at 8 weeks after the treatment.

### EXAMPLE 6

[Treatment of Major Depressive Disorder (Depression)]

[Method]

The combination drug of Formula antidoti coptidis (orengedokuto) and Formula glycyrrhizae atractylodis cinnamomi hoelen (ryokeijutsukanto) prepared by the method of Example 2 was used for the treatment of major depressive disorder. The combination drug was prepared by mixing 2.5 g of Tsumura Formula antidoti coptidis (orengedokuto) extract granule (ethical) (TSUMURA & CO.) and 2.5 g of Tsumura Formula glycyrrhizae atractylodis cinnamomi hoelen (ryokeijutsukanto) extract granule (ethical) (TSUMURA & CO.), and was administered three times a day before meal.

Treatment using the composition of the present invention was carried out for 187 cases of major depressive disorder (average age was 52.0 years old, 16 to 94 years old, male:female=46:141).

163 cases of major depressive disorder patients who wished to be treated with kampo drugs were treated with the composition of the present invention (kampo treatment group), and 24 cases of major depressive disorder patients who did not with to be treated with kampo drugs were treated as controls (control group). The use of antipsychotic drugs was not considered relevant. The cases were diagnosed under DSM-IV (Diagnostic Statistical Manual of Mental Disorders, Fourth Edition) and SDS (Zung Self-Rating Depression Scale). The remission of major depressive disorder was also judged according to DSM-IV and SDS. SDS was used for the observation before and after the treatment and follow-up, and objective difference was shown with numerical values. SDS of 40 or more than 40 is indicative of major depressive disorder. For the control group, anti-major depressive disorder medicine such as milnacipran hydrochloride, paroxetine hydrochloride hydrate, sertraline hydrochloride and sulpiride was administered for 83.3% patients (20/24). T-test and Yates 2×2 chi square test were conducted for statistical analysis.

[Results]

In the 183 cases of major depressive disorder, the complication rate of bronchial asthma, allergic rhinitis or allergic conjunctivitis was 88.8% (166/187). Dropout cases were 20 cases for the kampo treatment group which was 12.3% of the total patients. The average SDS for the kampo treatment group before the treatment was 52.8 (n=143), and the average SDS for the control group at the first observation was 52.6 (n=24). No statistical significant difference was observed at this time. The average SDS for the kampo treatment group after normalization was 33.3 (N=143) which decreased significantly (p<0.01). In the kampo treatment group, 97.2% (139/143) of patients were normalized by diagnosing using DMS-IV and SDS, and 2.1% of patients were not normalized. In the control group, 12.5% (3/24) of patients were normalized during the observation (average observation period was 42.7 weeks). Significant higher remission rate was observed (P<0.01) for the kampo treatment group than for the control group. The length till remission for the kampo treatment group was 9.7 weeks (n=139), and the average duration of administration was 20.2 weeks (n=139). Although recurrence was observed in 11 cases, all cases easily reached to remission. In one case of the kampo treatment group, there was a side effect in the form of a drug-induced liver disorder. The side effect was overcome with treatment discontinuation.

Nearly all cases of major depressive disorder were complicated by allergic complaints, and in those cases, both ailments were cured using kampo combination drug (Formula antidoti coptidis (orengedokuto) and Formula glycyrrhizae atractylodis cinnamomi hoelen (ryokeijutsukanto)). major depressive disorder is therefore considered to be an allergic or immune disorder, and this should lead to new methods of controlling major depressive disorder.

Given the global increase in major depressive disorder, and the difficulty in treating it, the present example shows that kampo drugs which can cure atopic dermatitis, bronchial asthma and allergic rhinitis can easily results in complete remission of major depressive disorder. This indicates that cause of major depressive disorder is enhancement of immune response or immunological inflammation, and the composition of the present invention can be used for new therapy for the complete remission of major depressive disorder.

### INDUSTRIAL AVAILABILITY

The composition comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components according to the present invention can be employed for preventing or treating allergic disease, which is atopic dermatitis, bronchial asthma or allergic rhinitis, or major depressive disorder.

1. A composition for preventing or treating an immune disease, comprising a mixture of Formula antidoti coptidis (orengedokuto) and Formula repletionis animalis et supletionis medii (hochuekkito) or a mixture of Formula antidoti coptidis (orengedokuto) and Formula glycyrrhizae atractylodis cinnamomi hoelen (ryokeijutsukanto) as active components.

2. A composition for preventing or treating an allergic disease, being the composition for preventing or treating an immune disease according to claim 1, wherein the immune disease is an allergic disease.

3. The composition for preventing or treating an allergic disease according to claim 2, wherein the allergic disease is atopic dermatitis.

4. The composition for preventing or treating an atopic dermatitis, being the composition for preventing or treating an allergic disease according to claim 3, comprising a mixture of Formula antidoti coptidis (orengedokuto) and Formula repletionis animalis et supletionis medii (hochuekkito) as active components, used for treating atopic dermatitis with inflammation of trunk or limbs, but without inflammation of face, ear or head.

5. The composition for preventing or treating an atopic dermatitis, being the composition for preventing or treating an allergic disease according to claim 3, comprising a mixture of Formula antidoti coptidis (orengedokuto) and Formula glycyrrhizae atractylodis cinnamomi hoelen (ryokeijutsukanto) as active components, used for treating atopic dermatitis with inflammation of face, ear or head, with or without inflammation of neck and trunk thereabove or limbs

6. The composition for preventing or treating an allergic disease, comprising a mixture of Formula antidoti coptidis (orengedokuto) and Formula glycyrrhizae atractylodis cinnamomi hoelen (ryokeijutsukanto) as active components according to claim 2, wherein the allergic disease is selected from the group consisting of bronchial asthma and allergic rhinitis.

7. The composition for treating major depressive disorder (depression), being the composition for treating immune disease, comprising a mixture of Formula antidoti coptidis (orengedokuto) and Formula glycyrrhizae atractylodis cinnamomi hoelen (ryokeijutsukanto) as active components according to claim 1, wherein the immune disease is major depressive disorder (depression).
